(19) United States
(12) Patent Application Publication
(10) Pub. No.: US 2014/0324183 A1
(43) Pub. Date: Oct. 30, 2014

(54) PROSTHETIC ACETABULAR CUP

(71) Applicants: Thorwald SPRINGER, Sheffield, Yorkshire (GB); Marek GRABOWSKI, Poznan (PL)

(72) Inventors: Thorwald Springer, Sheffield (GB); Marek Grabowski, Poznan (PL)

(21) Appl. No.: 14/359,250

(22) PCT Filed: Dec. 17, 2012
(86) PCT No.: PCT/GB2012/053159

§ 371 Date: May 19, 2014

(30) Foreign Application Priority Data
Dec. 15, 2011 (GB) 1121529.0

(51) Int. Cl. A61F 2/34; A61F 2/46

(52) U.S. Cl. CPC A61F 2/34; A61F 2/4609; A61F 2002/30616
USPC 623/22.24

(57) ABSTRACT

A prosthetic acetabular cup comprising a dome-shaped expandable shell having an annular rim portion (2) and an apex portion and a concave inner cavity extending therebetween for receiving a bearing liner; the shell further comprising an apex aperture (21) centrally disposed in the apex portion relative to a shell axis of rotation and a plurality of radially spaced slots (9) extending from proximal the rim portion to the apex aperture to define a plurality of shell segments expandable outwardly relative to the shell axis about a hinge portion connecting the segments together when an expanding force is applied at the aperture. The present invention provides a simple, correctable and mechanically stable means of inserting and securing a prosthetic acetabular cup in an acetabular bone cavity in a non-intrusive and precisely aligned manner. The present invention also provides a cup for cavities of different sizes/bone properties which further provides a stable and secure platform for an acetabular cup bearing liner to be mounted and secured in.

The present invention relates to total hip replacement joints and in particular to the acetabular cup used in such a joint.

A hip joint comprises a femoral part having a rounded femoral head which articulates in an acetabular cavity of the pelvis. Both of these parts may require replacing with a prosthetic implant as a result of osteoarthritis, injury or the like. A total hip replacement consists of replacing both the acetabular cavity and the femoral part. The prosthetic implant used in a total hip replacement consists of three parts; the acetabular cup, the femoral component and the prosthetic head.

The acetabular cup is the component which is placed into the acetabular cavity of the pelvis. Cartilage is removed from the acetabular cavity by a suitable reamer to expose cancellous bone and the acetabular cup is typically inserted and secured therein using friction or cement. The cup may be press-fitted into an acetabular cavity having an outer diameter which is slightly less than the cup itself to provide the fricton/interference fit. Alternatively, a cup corresponding in size with the acetabular cavity may be cemented in place. Further alternatively, the exterior surface of a cup may comprise a screw thread which engages with surrounding bone of the acetabular cavity or a cup may be fixed in place by one or more screws which engage the femoral bone when additional stability is required. Depending on the type of cup being used for a particular patient, the cup may be inserted and positioned using a suitable positioning tool, known as an introducer. Where the cup is a press-fit cup, it is then hammered into place to secure the same in the acetabular cavity.

Known acetabular cups typically include one piece plastic cups or metal modular cups. One piece cups are generally polyethylene and are cemented in the acetabular cavity. Cementless metal cups generally consist of two pieces; a shell and a liner. The outer surface of the metal shell comprises a coating to stimulate bone in-growth to help secure the cup in the acetabular cavity. The inside of the shell is designed to receive and support a bearing liner which may typically be polyethylene, ceramic or metal. Polyethylene liners are generally placed in the shell and connected thereto by a rim locking mechanism, whilst ceramic and metal liners are generally attached to the shell with a so-called ‘Morse taper’.

Total hip replacements are currently the most common orthopaedic operation. However, postoperative complications and problems can include dislocation, loosening, impingement, limited joint movement, joint wear (particularly of the polyethylene liner), infection, osteolysis, metal sensitivity and toxicity, nerve palsy, pain and leg length discrepancy. A known cause of some of these complications is misalignment of the acetabular cup which can occur during insertion and fixation, even when known navigational methods are used. It is known that optimum cup alignment is achieved in less than 50% of hip operations.

Misalignment is caused by the cup shell not being orientated correctly in the acetabular cavity of the pelvis which can be discovered during or after surgery. For example, hammering a press-fit cup into an acetabular cavity is not an accurate means of inserting the cup in the cavity, even if the surgeon was confident alignment had been achieved before insertion. Where misalignment has been discovered during surgery, the cup must be removed, the cavity re-reamed to remove further cartilage and a larger cup inserted, undesirably compromising the integrity of the acetabular cavity and being surgically time consuming. Where such misalignment is discovered after the original operation, revision surgery is undesirably required resulting in the integrity of the bone tissue bordering the cup being compromised, not to mention further inconvenience and increased surgical risks for the patient.

A first aspect of the present invention provides a prosthetic acetabular cup comprising a dome-shaped expandable shell having an annular rim portion and an apex portion and a concave inner cavity extending therebetween for receiving a bearing liner; the shell further comprising an apex aperture centrally disposed in the apex portion relative to a shell axis of rotation and a plurality of radially spaced slots extending from proximal the rim portion to the apex aperture to define a plurality of shell segments expandable outwardly relative to the shell axis about a hinge portion connecting the segments together when an expanding force is applied at the aperture.

Preferably the shell is non-hemispherical when in unexpanded form and is moved towards a hemispherical state when being expanded. Preferably the shell is close to non-hemispherical when in expanded form. The segments are forced to expand outwardly and equally relative to the shell axis about the hinge portion when an expanding force is applied at the aperture so that initial engagement between the shell and preformed bone cavity occurs proximal the hinge portion and is transferred gradually towards the apex portion of the shell during further expansion of the segments. To this effect, the initial pressure against the bone by the expansion of the segments is desirably spread gradually from the vicinity of the hinge portion to the apex portion of the shell. This desirably prevents ejection forces occurring which would otherwise tend to force the cup out of the cavity and enables interference fit forces to be distributed gradually and equally from the opening of the bone cavity towards its base as the shell expands.

The present invention thereby provides for precisely aligned insertion of the cup into a preformed acetabular bone cavity without the need for impact forces otherwise caused by hammering a press-fit cup, for example, which could cause misalignment of the cup and, where the cup needs realigning or replacing, the integrity of the cancellous bone to be compromised. The present invention further provides for equal and balanced transfer of forces from the cup to the bone during installation in the preformed acetabular cavity in an aligned and stable manner. Furthermore, if the shell is not aligned or orientated as desired, the expanding force may be removed from the aperture to allow the shell to elastically return to an unexpanded form. The shell can then be realigned/repositioned and expanded to secure the same in the acetabular cavity without the integrity of the underlying bone being compromised by such realignment/repositioning. This eliminates or at least significantly reduces the problems caused by misalignment occurring post operation which would otherwise undesirably require revision surgery. The present invention further provides a stable and secure platform for bearing liners.

Preferably the expanding force is provided by an expander comprising a conical element complimentarily shaped with the aperture to force the segments apart when inserted therein in an axial direction from the rim portion to the apex portion. Preferably the expander is a frustoconical element comprising an externally disposed screw thread corresponding to a screw thread of the aperture and is adapted to force the segments outwardly when screwed into the aperture.

Suitably the element comprises a throughbore or recess for a suitable tool, such as a screwdriver, Allen™ key or Torx™ drive for example, to engage with and drive the element into or out of the aperture. Preferably the element comprises a throughbore for a tool to engage and/or to view or feel the base of a preformed acetabular cavity to determine the depth of the cup in the cavity during or after installation. Suitably an introducer for initially inserting and aligning the shell in the acetabular cavity may detachably engage with the shell. Preferably such an introducer engages with the aperture or with the throughbore or recess of the element.

Preferably the element comprises an annular shoulder adapted to engage the shell when inserted in the aperture. Preferably the element and shoulder are adapted so that the shoulder completes the base of the shell cavity when the element is inserted in the aperture. Suitably an annular recess may surround the aperture with which the shoulder is complimentarily shaped to engage in. The shoulder and recess interface also suitably acts as a stop to limit the movement of the element in the aperture when a desired final position of the expanded segments has been reached.

Preferably the shoulder provides a locking mechanism between the element and the shell when fully inserted in the aperture. Suitably the annular recess may be bevelled to receive a complimentarily shaped shoulder of the element. A locking mechanism desirably prevents the element from loosening in the aperture post operation.

Suitably the element may be sized according to a desired amount of expansion for a particular acetabular cavity size. For example, an element of larger diameter will cause the shell segments to expand further to engage with a cavity of relatively large diameter compared with an element of smaller diameter which is more suited to a smaller acetabular cavity. The length of the element and/or screw thread characteristics, such as pitch, may also suitably be selected depending on the desired amount and nature of the expansion.

Preferably the slots extend longitudinally over a major part of the shell. Suitably the slots may comprise six primary slots being equally spaced by 60 degrees to each other about the shell axis. Such an arrangement suitably provides six shell segments hingedly connected together by the hinge portion. However, the number of slots and therefore number of segments may suitably be selected depending on the size of the shell and/or flexibility required.

Preferably each segment comprises a secondary slot disposed centrally therein and extending from the hinge portion to a point within limits of the segment. Preferably each secondary slot extends longitudinally relative to the shell axis and terminates at or near a midpoint of its corresponding segment. The secondary slots provide each segment with an additional degree of flexibility.

Preferably each primary and secondary slot comprises a first aperture from which it extends proximal the rim portion. Preferably the first apertures are radially aligned. Suitably the first apertures may define the hinge portion. The first apertures provide each segment with an additional degree of flexibility about the hinge portion. Suitably the first apertures may be circular holes or elongate slots. Preferably the first apertures are lateral slots relative to the shell axis. Suitably the first apertures may have an aspect ratio of about 2:1 in the sense of lateral length:longitudinal height, but of course other aspect ratios may be selected.

Suitably each secondary slot further comprises a secondary aperture at a point along its length. Preferably the secondary aperture of each secondary slot is provided at or near an end distal the first aperture. Preferably the secondary apertures are radially aligned. Such secondary apertures provide the shell segments with additional flexibility about the hinge portion.

Preferably the apex portion of the shell has a wall thickness which is greater than that of the hinge portion. Preferably the wall thickness of the shell increases gradually from the hinge portion to the apex portion. The reduced wall thickness provides the shell with increased flexibility and elasticity around the hinge portion.

Preferably the rim portion of the shell comprises an inwardly extending radial flange. Preferably the flange is adapted to receive and support a bearing liner.

Preferably an outer surface of the shell comprises a bone in-growth stimulating coating to encourage osseointegration.

A further aspect of the present invention provides a method of implanting an acetabular cup as described above in an acetabular cavity, comprising the steps of:
preforming the acetabular cavity using a suitable reamer;
selecting an acetabular cup from a plurality of cups in accordance with the present invention to correspond with the preformed cavity;
attaching the cup to one end of an introducer;
inserting and aligning the cup in the cavity;
applying the expanding force to the aperture to expand the shell segments outwardly to secure the cup in the cavity; and
detaching the introducer from the cup.

The method may further comprise one or more of the following steps:
providing an expander to cause the expanding force;
partially engaging the expander in the aperture before insertion of the cup in the cavity;
fully engaging the expander in the aperture to force the shell segments outwardly to secure the cup in the cavity;
disengaging the expander to retract the shell segments;
repositioning the shell in the cavity before re-engaging the expander in the aperture; and
providing drive means to engage with the expander and drive the same into or out of the aperture;
viewing the acetabular cavity through an aperture of the shell to determine the depth of the cup in the cavity during or after insertion; and
locking the expander in the aperture when the shell is fully expanded.

A further aspect of the present invention provides a prosthetic acetabular cup assembly comprising an acetabular cup as described above, an expander engageable in the central aperture of the shell, and a bearing liner mountable in said shell.

Suitably the expander may be selected from a plurality of differently sized expanders to provide different amounts of shell segment expansion depending on the cavity size and/or bone properties of the patient. Preferably the expander is a frustoconical screw element selected from a plurality of screw elements having different diameters and/or tapers and/or lengths.

The bearing liner may be a suitable liner of preferred size and material according to the shell size and femoral head properties respectively. Suitably the liner may be polyethylene, ceramic or metal. Suitably the liner is compatible with femoral head sizes ranging from preferably 26 mm to 36 mm in diameter, or other suitable size. Suitably the bearing liner may be selected from a plurality of differently sized liners to correspond with a selected shell size and/or femoral head size. Suitably the liner may be screwed into the shell or attached thereto by suitable means such as a locking mechanism.

A further aspect of the present invention provides a method of implanting a prosthetic acetabular cup assembly as described above in an acetabular cavity, the method comprising the steps of:
preforming the acetabular cavity using a suitable reamer;
attaching the cup to one end of an introducer;
inserting and aligning the cup in the cavity;
engaging the expander in the aperture to expand the shell segments outwardly to secure the cup in the cavity;
detaching the introducer from the cup; and
mounting the bearing liner in the shell.

Preferably the introducer is adapted to allow a suitable tool, such as screw driver or Allen key, to pass through the same and engage with the expander to drive the same into the aperture of the shell to expand the shell segments.

The method may further comprise one or more of the following steps:
selecting an acetabular cup from a plurality of cups to correspond with the preformed cavity;
selecting a bearing liner from a plurality of liners to correspond with the shell and/or a femoral head;
partially engaging the expander in the aperture before insertion into the cavity;
driving the expander with a suitable tool into the aperture to expand the shell segments outwardly;
disengaging the tool from the expander when the segments have been expanded to a desired position;
viewing the acetabular cavity through an aperture of the shell to determine the depth of the cup in the cavity during or after insertion; and
locking the expander in the aperture when the shell is fully expanded.

An embodiment of the invention will now be described by way of example only with reference to the accompanying drawings in which:

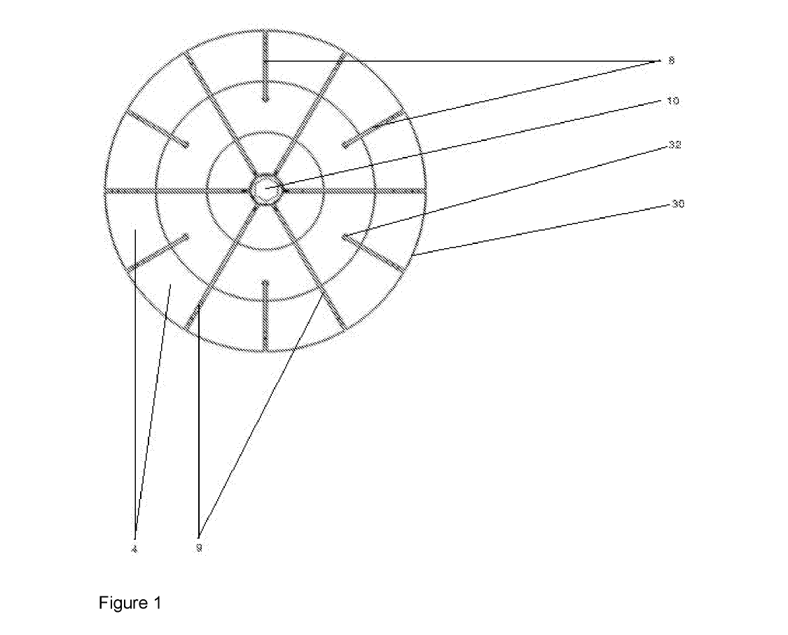

FIG. 1 shows an underside view of an acetabular cup in accordance with the present invention;

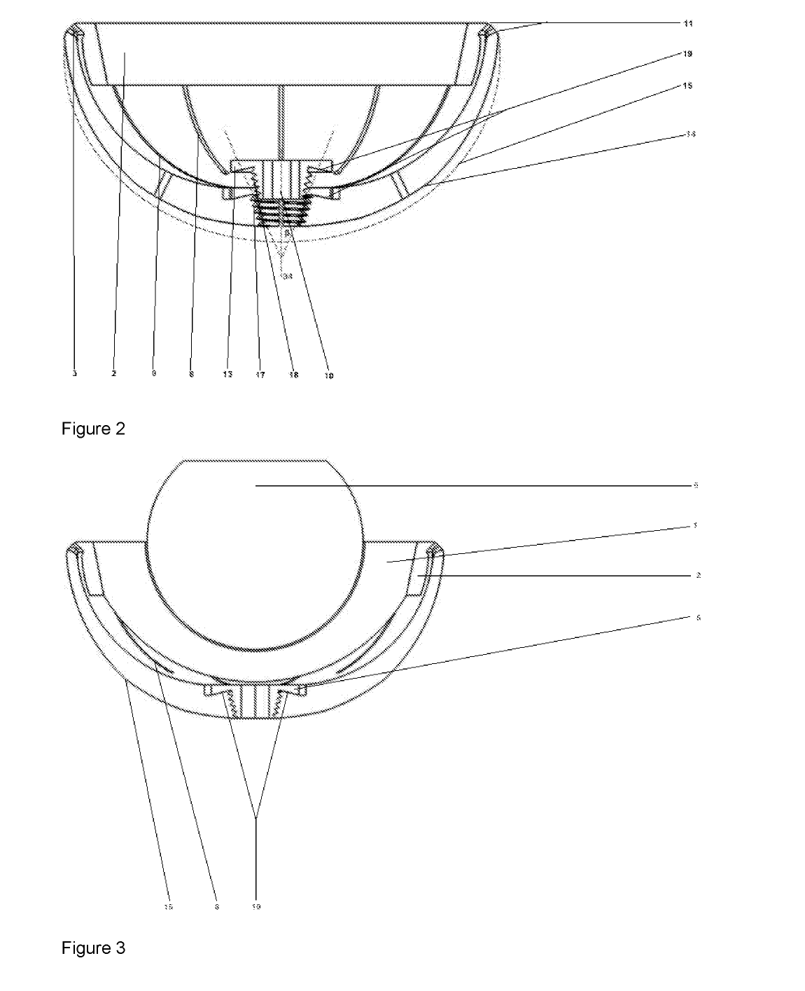

FIG. 2 shows a mid-section through the cup of FIG. 1 in retracted and expanded form;

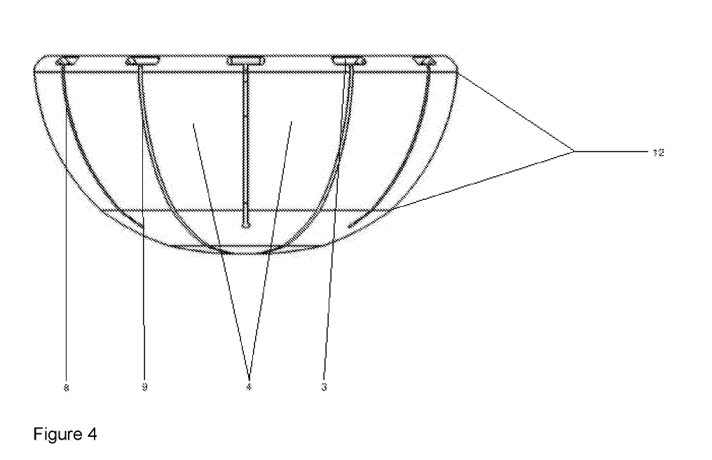

FIG. 3 shows an acetabular cup assembly including the cup of FIGS. 1 and 2 in expanded form and a bearing liner mounted therein for engagement with a prosthetic femoral head;

FIG. 4 shows side view of an acetabular cup in accordance with a present invention;

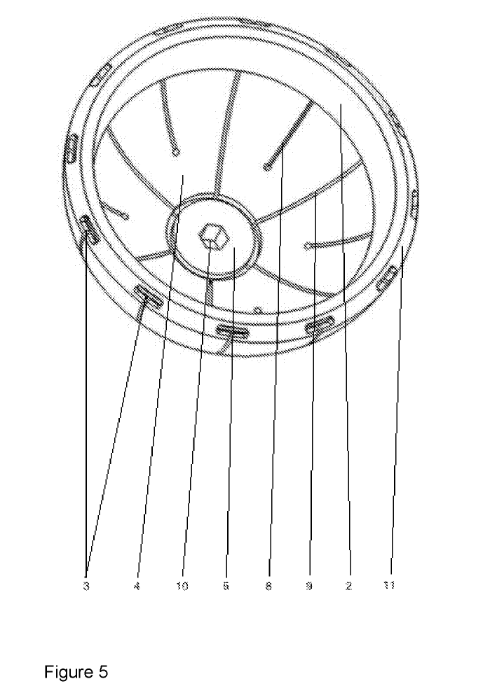

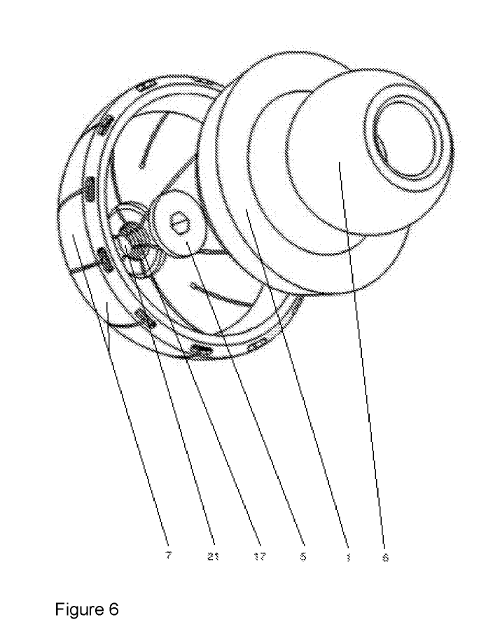

FIG. 5 shows acetabular cup in expanded form with a bearing liner removed showing frusto-conical element screwed in and locked;

FIG. 6 shows exploded view of acetabular cup, frusto-conical screw, bearing liner and prosthetic femoral head.

As shown in FIGS. 1 and 2, an acetabular cup comprises a dome-shaped shell having a rim portion 2 and a central aperture provided in an apex portion of the shell. The shell is formed of bio-compatible metal covered with a bone in-growth supporting coating 7. The outer surface is also roughened to enhance its security in an acetabular cavity and also to provide a key for bone in-growth. The shell is made up of six segments 4 defined by primary slots 9 and secondary slots 8 equally spaced and oriented along meridian planes of the shell. The slots 8 and 9 extend from primary apertures 3 in a sub-equatorial portion of the cup in the vicinity of the rim portion 2 towards the central aperture. The primary slots 9 terminate at the central aperture 21 whereas the secondary slots 8 terminate at secondary apertures 32 provided within the limits of the segments 4. The flexibility of the shell and movement of the segments is provided by slots 8 and 9 and primary apertures 3. The reducing and tapering thickness of the shell wall from the apex portion towards the rim portion provides additional flexibility and elasticity whilst ensuring the shell is sufficiently stiff when in expanded form.

As is further illustrated by the exploded view of FIG. 6, central aperture 21 comprises a tapered screw thread 17 to receiveably engage with frusto-conical element 5 comprising a corresponding screw thread 18. This arrangement provides an expanding force when screw 5 is driven into aperture 21 to expand the segments outwardly relative to a shell axis 34 of rotation about a hinge portion 11 defined by apertures 3. The conical screw 5 is provided with a central hole 10 correspondingly shaped to receive and engage with a standard Torx™/Allan™ tool (not shown). Outward expansion of the segments from the aperture about a hinge portion proximal the rim portion of the shell advantageously ensures initial engagement between the shell and a preformed bone cavity occurs proximal the hinge portion and is transferred gradually towards the apex portion of the shell during further expansion of the segments at elastic part of the shell 12 as shown in FIG. 4. To this effect, the initial pressure against the bone by the expansion of the segments is desirably spread gradually from the vicinity of the hinge portion to the apex portion of the shell. This desirably prevents ejection forces occurring which would otherwise tend to force the cup out of the cavity and enables interference fit forces to be distributed gradually and equally from the opening of the bone cavity towards its base as the shell expands. A mechanically stable acetabular cup for receiving a bearing liner is thereby provided.

As shown in FIG. 2, the shell is non-hemispherical when in retracted form, as shown by the solid lines 14, whilst being substantially hemispherical when the shell segments have been fully expanded, as shown by the dashed lines 15. Expansion of the segments is limited by a locking mechanism provided by bevelled shoulder 19 on the element 5 which correspondingly engages in bevelled recess 19 of the shell apex portion. Such a locking mechanism desirably ensures the element 5 does not loosen and disengage with the central aperture post operation causing shell segments to retract. The range of acetabular cup expansion and thus interference fit fixation grade can be modified depending on the specific application of the cup by selectively varying angle β of frusto-conical screw 5. The length and/or screw thread dimensions may also be selected depending on the amount and rate of expansion required for a particular cavity size and/or bone property. An element 5 may be selected from a plurality of differently sized elements to engage with a shell selected from a plurality of differently sized shells for engaging with differently sized cavities and/or receiving differently sized bearing liners.

FIG. 3 shows an acetabular cup in a fully expanded and locked position including an acetabular cup liner 1 suitably mounted on the rim portion 2 with an articulating prosthetic femoral head 6 engaged therein. As described above, the final, fully expanded position of the shell is defined by the frusto-conical screw 5 being fully screwed into the central aperture 21 and being secured by full engagement of the locking collar 13 into recess 19 of the shell. The above acetabular cup configuration is also shown in FIG. 5 with bearing liner removed showing frusto-conical screw fully engaged and locked.

The present invention provides a simple, correctable and mechanically stable means of inserting and securing a prosthetic acetabular cup in an acetabular bone cavity in a non-intrusive and aligned manner eliminating the need for high impact insertion forces and post operation corrective surgery. The present invention also provides a universal cup for cavities of different sizes/bone properties which further provides a stable and secure platform for an acetabular cup bearing liner to be mounted in.

1. A prosthetic acetabular cup comprising:
a dome-shaped expandable shell having an annular rim portion and an apex portion and a concave inner cavity extending therebetween for receiving a bearing liner; and
the shell further comprising an apex aperture centrally disposed in the apex portion relative to a shell axis of rotation and a plurality of radially spaced slots extending from proximal the rim portion to the apex aperture to define a plurality of shell segments expandable outwardly relative to the shell axis about a hinge portion connecting the segments together when an expanding force is applied at the aperture.

2. A prosthetic acetabular cup according to claim 1, wherein the shell is non-hemispherical when in unexpanded form and relatively close to hemispherical when in expanded form.

3. A prosthetic acetabular cup according to claim 1, wherein the expanding force is provided by an expander comprising a conical element complimentarily shaped with the aperture to force the segments apart when inserted therein in an axial direction from the rim portion to the apex portion.

4. A prosthetic acetabular cup according to claim 3, wherein the expander is a frustoconical element comprising an externally disposed screw thread corresponding to a screw thread of the aperture and is adapted to force the segments outwardly when screwed into the aperture.

5. A prosthetic acetabular cup according to claim 4, wherein the element comprises a throughbore or recess for a suitable tool to engage with and drive the element into or out of the aperture.

6. A prosthetic acetabular cup according to claim 5, wherein the throughbore provides means to view or feel the base of a preformed bone cavity to determine the depth of the cup in the cavity during or after installation.

7. A prosthetic acetabular cup according to claim 1, wherein the shell is adapted to detachably engage with an introducer for initially inserting and aligning the shell in the acetabular cavity.

8. A prosthetic acetabular cup according to claim 6, wherein the introducer is engagable with the aperture or with the throughbore or recess of the element.

9. A prosthetic acetabular cup according to claim 1, wherein the element comprises an annular shoulder adapted to engage the shell when inserted in the aperture.

10. A prosthetic acetabular cup according to claim 9, wherein the element and shoulder are adapted so that the shoulder completes the base of the shell cavity when the element is inserted in the aperture.

11. A prosthetic acetabular cup according to claim 10, wherein an annular recess surrounds the aperture with which the shoulder is complimentarily shaped to engage in.

12. A prosthetic acetabular cup according to claim 11, wherein the shoulder provides a locking mechanism between the element and the shell when fully inserted in the aperture.

13. A prosthetic acetabular cup according to claim 12, wherein the annular recess is bevelled to receive a complimentarily shaped shoulder of the element.

14. A prosthetic acetabular cup according to claim 1, wherein each segment comprises a secondary slot disposed centrally therein and extending from the hinge portion to a point within limits of the segment.

15. A prosthetic acetabular cup according to claim 14, wherein each secondary slot extends longitudinally relative to the shell axis and terminates at or near a midpoint of its corresponding segment.

16. A prosthetic acetabular cup according to claim 14, wherein each primary and secondary slot comprises a first aperture from which it extends proximal the rim portion.

17. A prosthetic acetabular cup according to claim 16, wherein the first apertures are radially aligned.

18. A prosthetic acetabular cup according to claim 16, wherein the first apertures are lateral slots relative to the shell axis.

19. A prosthetic acetabular cup according to claim 1, wherein the apex portion of the shell has a wall thickness which is greater than that of the hinge portion.

20. A prosthetic acetabular cup according to claim 19, wherein the wall thickness of the shell increases gradually from the hinge portion to the apex portion.

21. A prosthetic acetabular cup according to claim 1, wherein the rim portion of the shell comprises an inwardly extending radial flange for receiving and supporting a bearing liner.

22. A prosthetic acetabular cup according to claim 21, comprising a locking mechanism for locking a bearing liner in the shell.

23. A prosthetic acetabular cup according to claim 1, wherein an outer surface of the shell comprises a bone in-growth stimulating coating.

24. A method of implanting an acetabular cup according to claim 1 in an acetabular cavity, comprising the steps of:
preforming the acetabular cavity using a suitable reamer;
selecting an acetabular cup from a plurality of cups to correspond in size with the preformed cavity;
attaching the cup to one end of an introducer;
inserting and aligning the cup in the cavity;
applying the expanding force to the aperture to expand the shell segments outwardly to secure the cup in the cavity; and
detaching the cup from the introducer.

25. A method according to claim 24 comprising at least one of the following steps:
partially engaging the expander in the aperture before insertion of the cup in the cavity;
fully engaging the expander in the aperture to force the shell segments outwardly to secure the cup in the cavity;
disengaging the expander to retract the shell segments;
repositioning the shell in the cavity before re-engaging the expander in the aperture; and
providing drive means to engage with the expander and drive the same into or out of the aperture;
viewing the acetabular cavity through an aperture of the shell to determine the depth of the cup in the cavity during or after insertion; and
locking the expander in the aperture when the shell is fully expanded.

26. A prosthetic acetabular cup assembly comprising an acetabular cup according to claim 1, an expander engageable in the central aperture of the shell, and a bearing liner mountable in said shell.

27. A prosthetic acetabular cup assembly according to claim 26, wherein
the expander is a frustoconical screw element selected from a plurality of screw elements having different diameters and/or tapers and/or lengths and wherein the element is engageable with the central aperture of the shell comprising a corresponding screw thread.

28. A prosthetic acetabular cup assembly according to claim 26, wherein the liner is polyethylene, ceramic or metal.

29. A prosthetic acetabular cup assembly according to claim 28 wherein the bearing liner is selected from a plurality of differently sized liners to correspond with a selected shell size and/or femoral head size.

30. A method of implanting a prosthetic acetabular cup assembly according to claim 26 in an acetabular cavity, the method comprising the steps of:
preforming the acetabular cavity using a suitable reamer;
attaching the cup to one end of an introducer;
inserting and aligning the cup in the cavity;
engaging the expander in the aperture to expand the shell segments outwardly to secure the cup in the cavity;
detaching the introducer from the cup; and
mounting the bearing liner in the shell.

31. A method according to claim 30 wherein the introducer is adapted to allow a suitable tool to pass through the same and engage with the expander to drive the same into the aperture of the shell to expand the shell segments.

32. A method according to claim 30 further comprising one or more of the following steps:
selecting an acetabular cup from a plurality of cups to correspond with the preformed cavity;
selecting a bearing liner from a plurality of liners to correspond with the shell and/or a femoral head;
partially engaging the expander in the aperture before insertion into the cavity;
driving the expander with a suitable tool into the aperture to expand the shell segments outwardly;
disengaging the tool from the expander when the segments have been expanded to a desired position;
viewing the acetabular cavity through an aperture of the shell to determine the depth of the cup in the cavity during or after insertion; and
locking the expander in the aperture when the shell is fully expanded.
